(19) United States
(12) Patent Application Publication
(10) Pub. No.: US 2007/0260306 A1
(43) Pub. Date: Nov. 8, 2007

(54) Ophthalmic devices and procedures

(75) Inventor: Terence Arnold Waldock, Bedfordshire (GB)

Correspondence Address: SEED INTELLECTUAL PROPERTY LAW GROUP PLLC, 701 FIFTH AVENUE, SUITE 5400, SEATTLE, WA, 98104-7092, US

(73) Assignee: Duckworth & Kent Limited, Hertfordshire (GB)

(21) Appl. No.: 11/715,286

(22) Filed: Mar. 6, 2007

Related U.S. Application Data
(63) Continuation of application No. 10/487,960, filed on Aug. 4, 2004, now abandoned.

(30) Foreign Application Priority Data
Aug. 31, 2001 (GB) 0121169.7
Feb. 16, 2002 (GB) 0203712.5
Jun. 15, 2002 (GB) 0213846.9

(51) Int. Cl. A61F 2/14

(52) U.S. Cl. 623/4.1

(57) ABSTRACT

A tissue barrier (410), for example of titanium alloy, for insertion into an incision in the sclera of the eye for the treatment of conditions such as presbyopia, comprises an elongate insert which is provided with a longitudinally extending flange (414, 416) at each end to prevent the barrier from being ejected from the incision. Each flange (414, 416) is preferably about half the height of the central portion (412) of the insert. Preferably, the flanges are of unequal length. The flanges may be circular or rectangular in cross-section. The central portion (412) may be thicker than the flanges transversely to the longitudinal axis of the insert. The outer ends of the flanges may be chamfered (418).

### FIELD OF THE INVENTION

This invention relates to ophthalmic devices and procedures, and is particularly concerned with the treatment of presbyopia.

### BACKGROUND TO THE INVENTION

It is known to make radial incisions extending into the anterior ciliary-area of the sclera. Such techniques were used in the early 1980s as part of radial keratotomy procedures. One disadvantage of this procedure was that it could produce a myopic shift. Any procedure which improves the working ability of the ciliary body/zonular complex will enhance accommodation of the eye. Therefore, the use alone of incisions into the sclera will increase the working circumference and enhance accommodation. However, in the healing process, new blood vessels and collagen across the width of the incisions contract as they heal. This results in shrinkage and scarring, and the effect of the original incisions is reduced.

Various attempts have been made to treat presbyopia. In U.S. Pat. No. 5,489,299 the amplitude of accommodation of the eye is increased by manipulating the ciliary muscle through intervention with external means. This can be accomplished by securing a rigid band to the portion of the sclera which radially surrounds the ciliary body.

In U.S. Pat. No. 5,722,952, the sclera is weakened by use of an enzyme which can be injected into the sclera in the region of the ciliary body.

In U.S. Pat. No. 5,354,331 the effective working distance of the ciliary muscle is increased by expanding the sclera in the region of the ciliary body. This is accomplished by suturing to the sclera in the region of the ciliary body a relatively rigid band having a diameter slightly greater than that of the sclera in that region. The scleral expansion band comprises anterior and posterior rims and a web extending between the rims.

### SUMMARY OF THE INVENTION

It is an object of the present invention to enable an effective treatment of presbyopia to be effected, by maintaining the effect of incisions made into the sclera and preventing subsequent shrinkage such as has previously taken place due to the natural healing process.

This is achieved in accordance with the invention by the insertion of tissue barriers into the incisions made in the sclera. A tissue barrier inserted into the sclera will keep the incision open and healing will take place without fibrosis. The tissue barrier will prevent in-growth of tissue. The tissue barriers prevent the contracting which would otherwise occur as part of the healing process, and allow the internal pressure of the eye, i.e. the intraocular pressure, to maintain the stretching which is produced by the original incisions, and thus correcting the presbyopic condition.

It is important to note that the tissue barriers of the present invention are not “implanted” in the sense of being buried in channels created by tunnelling through the sclera. The tissue barriers are placed in the incisions made in the sclera, simply as a barrier to in-growth of new tissue. It is the internal hydrodynamics of the eye, i.e. the constant intraocular pressure, which then effectively stretches the sclera, allowing for increased tension in the ciliary body/zonular complex. The use of the tissue barriers in accordance with the invention creates a passive increase in the intraocular pressure.

The tissue barriers are placed in four or more incisions in the sclera. The incisions increase the effective working distance of the ciliary muscle, by increasing the radial distance between the crystalline lens and the inner diameter of the ciliary muscle. No “segments” are inserted as none are necessary. The tissue barriers are used simply to maintain the integrity of the incisions and allow continued stretching by internal ocular pressure. There is no manipulation of the ciliary muscle involved in the procedures according to the present invention. The tissue barriers serve only to maintain the integrity of the incisions.

In accordance with one aspect of the present invention there is provided a tissue barrier for insertion into an incision in the sclera of the eye, comprising an elongate insert which is provided with a longitudinally extending flange at each end to prevent the barrier from being ejected from the incision.

In contrast to an insert which has an inverted T cross-section and lateral flanges to prevent its ejection as healing takes place, the use of longitudinally extending flanges at each end of the insert has practical advantages in terms of making the operating procedure easier. Making a T-shaped incision is not easy. The cutting of lateral incisions after making the initial radial incision is difficult to achieve, both because of the surgical instruments needed and the fact that the sclera varies in dimensions from eye to eye. By making the radial incision in just the one plane, the procedure is simplified, while the end flanges still hold the insert in place both initially and during the healing process.

Although the tissue barriers can be of any material, barriers of titanium or a titanium alloy are particularly preferred.

Preferably, the insert has a central longitudinally extending portion with a reduced height flange at each end, the bottom surface of each flange being contiguous with the bottom surface of the central portion.

In a preferred embodiment, the height of each flange is about half the height of the central portion.

In some embodiments, the flanges at the respective ends of the central portion are not of equal length.

The flanges desirably have a circular or rectangular cross-section.

In some embodiments, the central portion has a greater width transversely to the longitudinal axis of the insert than the flanges.

In some practical embodiments, the dimension of each flange transversely to the longitudinal axis of the insert is of the order of 0.20 mm, and the flanges are respectively of the order of 0.90 mm and 0.50 mm in length.

The overall length of the insert is preferably between of the order of 4.25 mm and of the order of 3.75 mm.

Preferably, the combined length of the two flanges is approximately equal to one third of the overall length of the tissue barrier.

Also in accordance with the invention there is provided a method of treating presbyopia, which comprises making a plurality of radial incisions in the sclera of the eye, at the base of each incision making longitudinal extensions of the incision in each longitudinal direction, and inserting a tissue barrier of the type defined above into each incision so as to prevent the tissue barrier being ejected.

### BRIEF DESCRIPTION OF THE DRAWINGS

In order that the invention may be more fully understood, a number of embodiments of tissue barrier in accordance with the invention will now be described by way of example and with reference to the accompanying drawings. In the drawings:

FIG. 1 is a top plan view of a first embodiment of tissue barrier in accordance with the invention;

FIG. 2 is a side view of the tissue barrier of FIG. 1;

FIG. 3 is an end view of the tissue barrier of FIGS. 1 and 2;

FIG. 4 is a side view of a second embodiment of tissue barrier in accordance with the invention;

FIG. 5 is an end view of the tissue barrier of FIG. 4;

FIG. 6 is a top plan view of the tissue barrier of FIGS. 4 and 5;

FIG. 7 is a side view of a third embodiment of tissue barrier in accordance with the invention;

FIG. 8 is an end view of the tissue barrier of FIG. 7;

FIG. 9 is a top plan view of the tissue barrier of FIGS. 7 and 8,

FIG. 10 is a side view of a fourth embodiment of tissue barrier in accordance with the invention;

FIG. 11 is an end view of the tissue barrier of FIG. 10;

FIG. 12 is a top plan view of the tissue barrier of FIGS. 10 and 11;

FIG. 13 is a side view of a fifth embodiment of tissue barrier in accordance with the invention;

FIG. 14 is an end view of the tissue barrier of FIG. 13;

FIG. 15 is a top plan view of the tissue barrier of FIGS. 13 and 14;

FIG. 16 is a side view of a sixth embodiment of tissue barrier in accordance with the invention;

FIG. 17 is an end view of the tissue barrier of FIG. 16; and,

FIG. 18 is a top plan view of the tissue barrier of FIGS. 16 and 17.

### DESCRIPTION OF THE PREFERRED EMBODIMENTS

The tissue barriers shown in the drawings are preferably made of a titanium alloy, although other materials could be used.

In the drawings all the indicated dimensions are given in millimetres (mm).

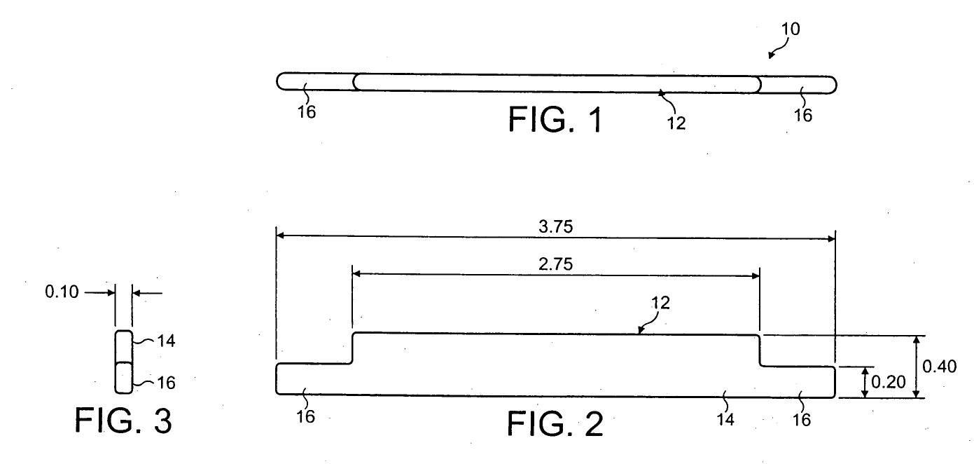

Referring first to FIGS. 1 to 3 of the drawings, the tissue barrier 10 comprises a thin plate 12 which has a central, longitudinally extending portion 14 and a reduced height flange 16 at each end. The bottom surface of each flange is contiguous with the bottom surface of the central portion 14. As shown in the drawing, a preferred embodiment of tissue barrier has an overall length of 3.75 mm, a length for the central portion 14 of 2.75 mm, a length of each flange 16 of 0.50 mm, a thickness of 0.10 mm, an overall height of 0.40 mm and a height of each flange of 0.20 mm. In other words, the height of each flange 16 is approximately half the overall height of the tissue barrier. This provides sufficient stability for the tissue barrier to be held in place within the incision in the sclera.

Because the sclera varies from eye to eye, an overall height of the tissue barrier of 0.40 mm is considered the optimum dimension.

In order to insert the tissue barrier into the sclera, the surgeon will first make a radial incision 3 mm in length in the sclera. This is just slightly longer than the length of the centre portion 14 of the tissue barrier plate. Then, using a blade having a suitably shaped head, the surgeon can insert the cutter to the base of the incision and push it forwards and backwards to cut pockets at each end of the main radial incision, into which the flanges 16 will be received. A cut of just 0.5 mm at each end of the main radial incision is sufficient. This is a simple procedure. The tissue barrier 10 can then be inserted into the incision, by retracting the sclera and springing the tissue barrier into place, first locating the flange 16 at one end in its pocket and then locating the flange 16 at the other end within its pocket.

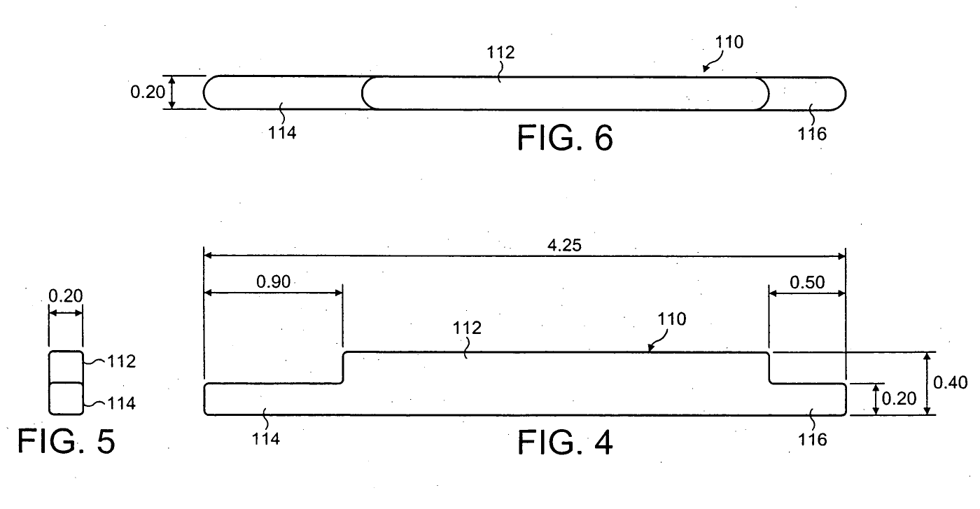

Referring now to the tissue barrier shown in FIGS. 4 to 6, this tissue barrier 110 comprises an element of titanium or titanium alloy which has a central, longitudinally extending portion 112 and reduced height flanges 114 and 116 at each end. The flange 114 at one end of the tissue barrier is longer than the flange 116 at the other end of the tissue barrier. This is to facilitate insertion of the tissue barrier into a pocket created by an incision. The longer flange 114 is first inserted into the pocket, and when that is properly seated the shorter flange 116 can be pushed into place more easily than if it was of the same length as the longer flange. The flange lengths are still sufficient to ensure that the tissue barrier is held in place within the sclera without being ejected. As shown in the drawing, a preferred embodiment of tissue barrier has an overall length of 4.25 mm, a length for the central portion 112 of 2.85 mm, a length for the longer flange 114 of 0.90 mm and a length for the shorter flange 116 of 0.50 mm. The tissue barrier has a thickness of 0.20 mm. The overall height of the tissue barrier is 0.40 mm, with the height of each flange 114, 116, being approximately half the overall height of the barrier. As shown in FIG. 5, the flanges are of substantially square cross-section and the central portion 112 is of substantially rectangular cross-section, although with the corners slightly rounded to remove any sharp edges. As shown in FIG. 6, the end faces of both the central portion 112 and of the two flanges 114 and 116 are rounded, again to facilitate the insertion of the tissue barrier into the pocket.

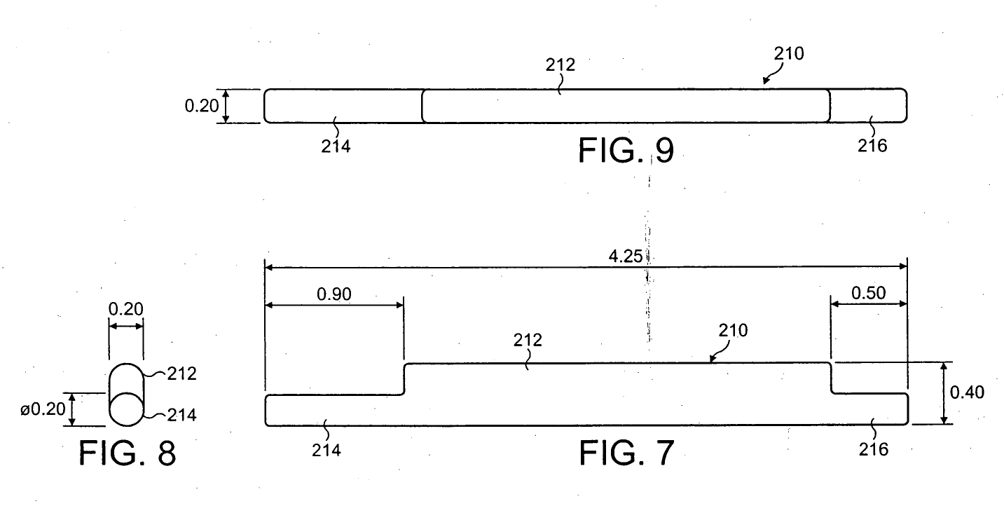

Referring now to the embodiment of tissue barrier shown in FIGS. 7 to 9, and which is indicated generally at 210, this has the same overall dimensions in terms of length as the tissue barrier of FIGS. 4 to 6. However, in this embodiment, the end flanges 214 and 216 are of circular cross-section, as can be seen from FIG. 8. The central portion 212 is rounded at the top and bottom, again as shown in FIG. 8. The central section 212 therefore has parallel sides with part-circular surfaces at top and bottom. In contrast to the embodiment shown in FIGS. 4 to 6, the end faces of the flanges and of the central portion are not radiused but are generally flat, as shown in FIG. 9.

In order to insert the tissue barrier of either of these embodiments into the sclera, the surgeon will first make a radial incision in the sclera using dissecting forceps. The dissecting forceps have a cutting head which is dimensioned to match the shape of the tissue barrier, i.e. with a first cutting element extending in one direction being longer than a second cutting element which extends in the other direction. Then, holding the tissue barrier by the shorter flange using insertion forceps, the surgeon will retract the sclera and lower the tissue barrier into place, first locating the longer flange 114, 214 in the pocket and then locating the shorter flange 116, 216 within the pocket.

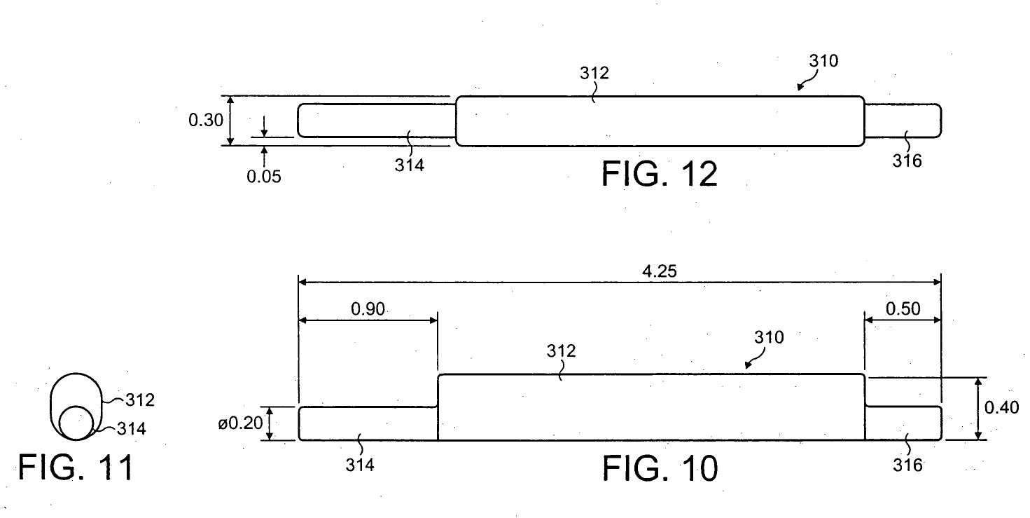

Referring next to the tissue barrier shown in FIGS. 10 to 12, this tissue barrier 310 comprises an element of titanium or titanium alloy which has a central, longitudinally extending portion 312 and reduced height flanges 314 and 316 at each end. The flange 314 at one end of the tissue barrier is longer than the flange 316 at the other end of the tissue barrier. This is to facilitate insertion of the tissue barrier into a pocket created by an incision, as explained above. As shown in the drawing, a preferred embodiment of tissue barrier has an overall length of 4.25 mm, a length for the central portion 312 of 2.85 mm, a length for the longer flange 314 of 0.90 mm and a length for the shorter flange 316 of 0.50 mm. The overall height of the tissue barrier is 0.40 mm, with the depth of each flange 314, 316 being approximately half the overall height of the barrier.

As shown in FIG. 12, the thickness of the tissue barrier, transversely to the longitudinal axis, is 0.30 mm, with the thickness of the flanges 314 and 316 being 0.20 mm.

As shown in FIG. 11, the flanges 314 and 316 are of circular cross-section and the central portion 312 is rounded at the top and the bottom, so as to have parallel sides with part-circular surfaces at top and bottom.

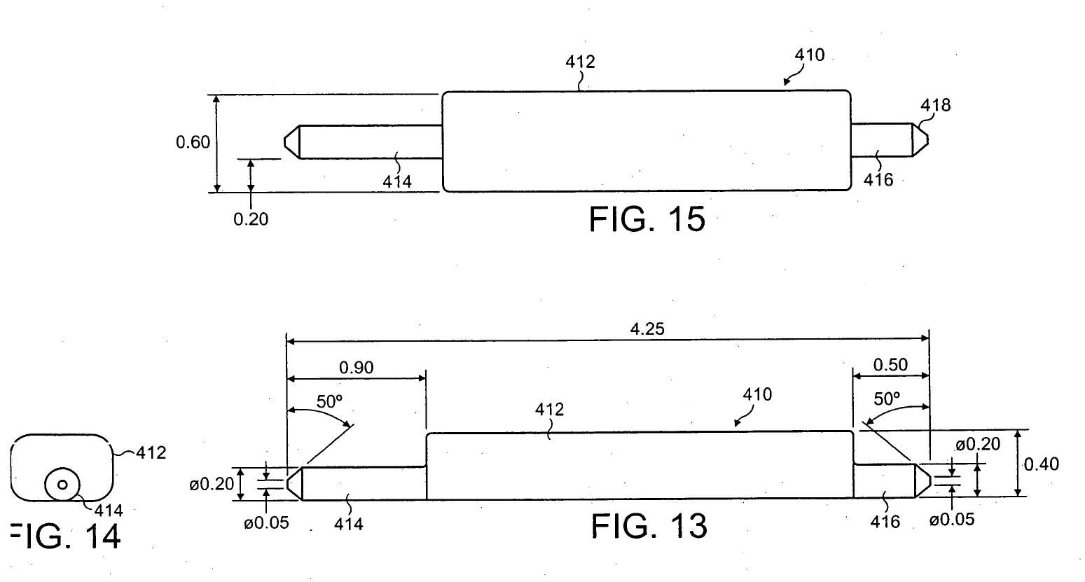

Referring now to the embodiment of tissue barrier shown in FIGS. 13 to 15, and which is indicated generally at 410, this has the same overall dimensions in terms of length as the tissue barrier of FIGS. 10 to 12. The end flanges 414 and 416 are also the same length as in the preceding embodiment. The flanges 414 and 416 are also circular in cross-section in this embodiment but are chamfered at each end as indicated at 418 in order to facilitate the insertion of the tissue barrier into the incision in the sclera. The taper angle is shown as 50°. It is to be noted that the provision of a chamfer at the outer ends of the flanges is a feature which could be used in any of the embodiments described above.

In the embodiment shown in FIGS. 13 to 15 the thickness of the tissue barrier is substantially greater, namely 0.60 mm. The thickness of each flange 414 and 416 is one-third of the thickness of the central portion 412 of the tissue barrier, i.e. 0.20 mm. As shown in FIG. 14, the central portion 412 has a substantially rectangular cross-section, although with the corners slightly rounded to remove any sharp edges.

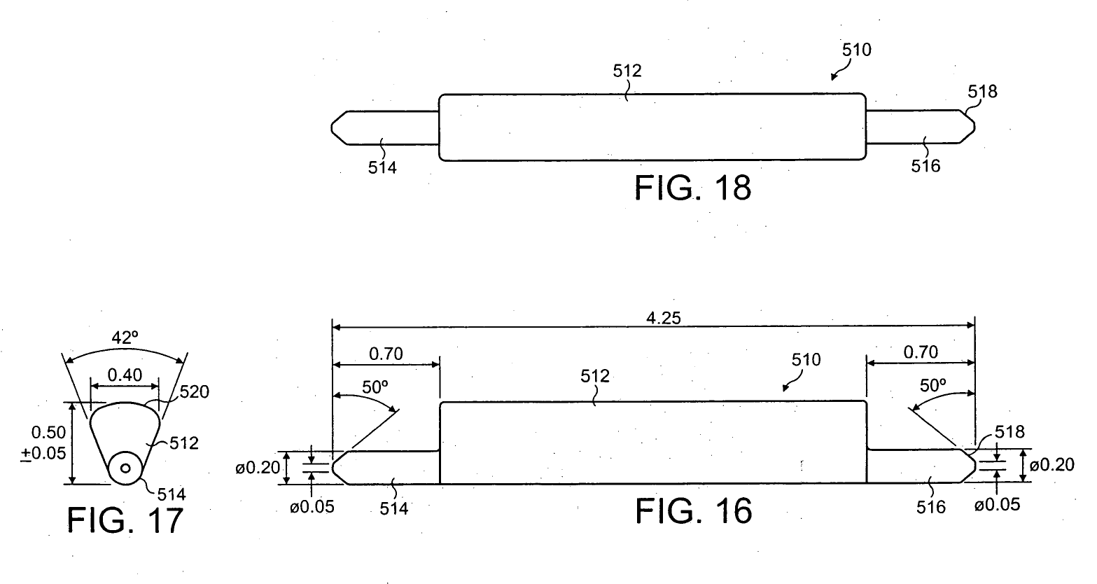

FIGS. 16 to 18 show a further embodiment of tissue barrier in accordance with the invention. In this embodiment the two flanges 514 and 516, are of equal length, namely 0.70 mm. The outer end of each flange is chamfered as indicated at 518. However, in this embodiment, the central portion 510 is not of constant thickness in the vertical direction but, as shown most clearly in FIG. 17, is sector-shaped. The lower part of the central portion 512 conforms to the shape of the end flanges, but the central portion broadens out in an upward direction and has an arcuate upper surface 520. In contrast to the embodiments described above, where the height of the flanges is approximately one half the height of the central portion, in this embodiment the ratio of the height of the flanges to the height of the central portion is approximately 2 to 5. The sector angle of the central portion 512 is 42°, although other angles of that order of magnitude could alternatively be used.

In this embodiment, as in the earlier embodiments, although the flanges 514 and 516 are of equal length, the combined length of the two flanges is approximately equal to one-third of the overall length of the tissue barrier.

1. A tissue barrier for insertion into an incision in a sclera of an eye, comprising an elongate insert which is provided with a longitudinally extending flange at each end to prevent the barrier from being ejected from the incision, the flanges having a reduced height as compared to the central portion between the flanges.

2. The tissue barrier as claimed in claim 1, each flange having a bottom surface contiguous with a bottom surface of the central portion.

3. The tissue barrier as claimed in claim 1, in which each flange has a height that is about half the height of the central portion.

4. The tissue barrier as claimed in claim 1, in which the flanges at the ends of the central portion are not of equal length.

5. The tissue barrier as claimed in claim 1, in which the flanges have a substantially circular cross-section.

6. The tissue barrier as claimed in claim 2, in which the flanges have a substantially circular cross-section and the central portion has rounded upper and lower surfaces.

7. The tissue barrier as claimed in claim 1, in which the flanges have a substantially rectangular cross-section.

8. The tissue barrier as claimed in claim 2, in which the flanges and the central portion have a substantially rectangular cross-section.

9. The tissue barrier as claimed in claim 2, in which the central portion has a greater width transversely to a longitudinal axis of the insert than the flanges.

10. The tissue barrier as claimed in claim 2, in which the central portion has a greater width transversely to a longitudinal axis of the insert than the flanges, the central portion has a substantially rectangular cross-section, and the flanges have a substantially circular cross-section.

11. The tissue barrier as claimed in claim 10, in which each flange has a width that is approximately one third of the width of the central portion.

12. The tissue barrier as claimed in claim 1, in which the dimension of each flange transversely to the longitudinal axis of the insert is of the order of 0.20 mm.

13. The tissue barrier as claimed in claim 1, in which one flange has a length of the order of 0.90 mm and the other flange has a length of the order of 0.50 mm.

14. The tissue barrier as claimed in claim 1, in which each flange has a length of the order of 0.70 mm.

15. The tissue barrier as claimed in claim 2, in which the central portion of the insert between the flanges has a height of the order of 0.40 mm.

16. The tissue barrier as claimed in claim 14, in which the height of the central portion of the insert between the flanges is of the order of 0.50 mm.

17. The tissue barrier as claimed in claim 2, in which the insert has an overall length between of the order of 4.25 mm and of the order of 3.75 mm.

18.-22. (canceled)

23. The tissue barrier as claimed in claim 1, in which each flange has an outer end that is chamfered so as to taper to a reduced cross-section at the outer end of the flange.

24. The tissue barrier as claimed in claim 1, made of titanium or a titanium alloy.

25. A method of treating presbyopia, which comprises making a plurality of radial incisions in a sclera of an eye, making longitudinal extensions of the incision in each longitudinal direction at the base of each incision, and inserting a tissue barrier as claimed in claim 1 into each incision.
